(19) United States
(12) Patent Application Publication
(10) Pub. No.: US 2022/0331333 A1
(43) Pub. Date: Oct. 20, 2022

(54) RHOA PATHWAY INHIBITOR IN THE TREATMENT OF NEURODEVELOPMENTAL DISORDER OR NEUROPSYCHIATRIC DISORDER

(71) Applicants: UNIVERSITE DE STRASBOURG, Strasbourg (FR); INSTITUT NATIONAL DE LA SANTE DE LA RECHERCHE MEDICALE (INSERM), Paris (FR); CENTRE NATIONAL DE LA RECHERCHE SCIENTIFIQUE (CNRS), Paris (FR)

(72) Inventors: Yann HERAULT, Illkirch (FR); Sandra MARTIN LORENZO, Strasbourg (FR)

(21) Appl. No.: 17/719,776

(22) Filed: Apr. 13, 2022

Related U.S. Application Data
(60) Provisional application No. 63/175,630, filed on Apr. 16, 2021.

(51) Int. Cl. A61K 31/551

(52) U.S. Cl. CPC A61K 31/551

(57) ABSTRACT

The present invention relates to the prevention and/or treatment of a neurodevelopmental disorder or a neuropsychiatric disorder comprising administering a therapeutically effective amount of fasudil or a derivative thereof or a pharmaceutical composition comprising fasudil or a derivative thereof, in a subject in need thereof.

### RELATED APPLICATIONS

The present application claims priority to U.S. Provisional Patent Application No. 63/175,630, filed Apr. 16, 2021, which is incorporated by reference herein in its entirety.

### FIELD OF THE INVENTION

The present invention concerns the prevention and/or treatment of a neurodevelopmental disorder or a neuropsychiatric disorder comprising administering a therapeutically effective amount of fasudil or a derivative thereof or a pharmaceutical composition comprising fasudil in a subject in need thereof.

### BACKGROUND OF THE INVENTION

People with neurodevelopmental disorders can experience difficulties with language and speech, motor skills, behavior, memory, learning, or other neurological functions. While the symptoms and behaviors of neurodevelopmental disabilities often change or evolve as a child grows older, some disabilities are permanent. Genetics can play an important role in many neurodevelopmental disorders, and in some cases is associated with specific genes. However, most neurodevelopmental disorders have complex and multiple contributors rather than any one clear cause. These disorders likely result from a combination of genetic, biological, psychosocial and environmental risk factors.

Neurodevelopmental disorders include Autism spectrum disorders (ASDs). ASDs are a group of developmental disabilities defined by significant social, communication, and behavioral impairments. The term “spectrum disorders” refers to the fact that although people with ASDs share some common symptoms, ASDs affect different people in different ways. ASDs encompass autistic disorder and the generally less severe forms, Asperger's syndrome and pervasive developmental disorder-not otherwise specified (PDD-NOS). Children with ASDs may lack interest in other people, have trouble showing or talking about feelings, and avoid or resist physical contact. Another hallmark characteristic of ASDs is the demonstration of restrictive or repetitive interests or behaviors, such as lining up toys, flapping hands, rocking their body, or spinning in circles.

Still today, diagnosis and treatment of neurodevelopmental disorders can be difficult; treatment often involves a combination of professional therapy, pharmaceuticals, and home- and school-based programs. New treatments are still needed in order to improve the everyday life of people suffering from these disorders.

Among genetic risks factors, gene copy number variants (CNV) have an important role in the appearance of neurodevelopmental disorders. Particularly, the deletion of the 16p11.2 locus is associated with autism spectrum disorder, intellectual disability, and several other features neuropsychiatric disorders, such as epilepsy, attention deficit hyperactivity disorder, schizophrenia and bipolar disorder. Earlier studies highlighted the implication of Kctd13 genetic imbalance in the 16p11.2 deletion.

The inventors discovered that targeting the Ras homolog family member A (RHOA) pathway rescue the cognitive phenotypes of the 16p11.2 deletion in mouse models. Indeed, modeling of 16p11.2 rearrangements in model organisms recapitulates the impairments present in humans including hyperactivity, repetitive behaviors and recognition memory deficits. In particular, inventors used chronic administration of fasudil (HA1077), an inhibitor of the Rho-associated protein kinase (ROCK), in mouse models carrying a heterozygous inactivation of Kctd13, or the deletion of the entire 16p11.2 BP4-BP5 region. They showed that a chronic treatment with fasudil can restore object recognition memory in both mouse models but does not change other behavioral traits. These findings confirm the pertinence of the RHOA pathway as a therapeutic path for the treatment of neurodevelopmental or neuropsychiatric disorders caused by the 16p11.2 deletion.

### SUMMARY OF THE INVENTION

The present invention concerns a method of preventing or treating a neurodevelopmental disorder or a neuropsychiatric disorder in a subject, comprising administering a therapeutically effective amount of fasudil or a derivative thereof or a pharmaceutical composition comprising fasudil or a derivative thereof, in a subject in need thereof.

In an embodiment, said disorder is selected among intellectual disability, autism spectrum disorder, epilepsy, attention deficit/hyperactivity disorder, schizophrenia and bipolar trouble, in a preferred embodiment the disorder is an autism spectrum disorder.

In a particular embodiment, the subject suffers from hyperactivity, repetitive behaviors and/or deficit in object memory.

In a particular embodiment, the subject suffers from a disorder associated with the rearrangement or the deletion of the 16p11.2 locus.

In a further embodiment, the subject does not exhibit an increased level of RHOA in the hippocampal region.

In a particular embodiment, the method according to the invention improves cognition. In a specific embodiment, the method according to the invention improves novel object recognition.

In a particular embodiment, fasudil or the derivative thereof is administered orally. In another particular embodiment, fasudil or the derivative thereof is administered on a daily basis.

In another particular embodiment, the subject is a mammal, more preferably a human.

The present invention also concerns the use of fasudil or a derivative thereof for preventing or treating a neurodevelopmental disorder or a neuropsychiatric disorder in a subject.

In an embodiment, said disorder is selected among intellectual disability, autism spectrum disorder, epilepsy, attention deficit/hyperactivity disorder, schizophrenia, and bipolar trouble, in a preferred embodiment, the disorder is an autism spectrum disorder.

In a particular embodiment, the subject suffers from hyperactivity, repetitive behaviors and/or deficit in object memory.

In a particular embodiment, the subject suffers from a disorder associated with the rearrangement or the deletion of the 16p11.2 locus.

In a further embodiment, the subject does not exhibit an increased level of RHOA in the hippocampal region.

In a particular embodiment, the use of fasudil or a derivative thereof according to the invention improves cognition. In a specific embodiment, the use of fasudil or a derivative thereof according to the invention improves novel object recognition.

In a particular embodiment, fasudil or the derivative thereof is administered orally. In another particular embodiment, fasudil or the derivative thereof is administered on a daily basis.

In another particular embodiment, the subject is a mammal, more preferably a human.

In a particular embodiment, a pharmaceutical composition for preventing or treating a neurodevelopmental disorder or a neuropsychiatric disorder in a subject, comprising fasudil or a derivative thereof is provided.

In another particular embodiment, the pharmaceutical composition comprises fasudil or the derivative thereof, and a pharmaceutically acceptable carrier.

### DETAILED DESCRIPTION OF THE INVENTION

### Definitions

In the context of the invention, the term “treating” or “treatment” means reversing, alleviating, inhibiting the progress of the disorder or condition to which such term applies, or one or more symptoms of such disorder or condition.

In the context of the invention, the term “preventing” or “prevention” refers to the prophylactic treatment of a subject who is at risk of developing a condition resulting in a decrease in the probability that the subject will develop the condition or a delay in the development of the condition.

In the context of the invention, the term “subject” refers to a mammal, such as a rodent, a feline, a canine, or a primate. Preferably, a subject according to the invention is a human.

Fasudil (HA1077, CAS number 103745-39-7, C₁₄H₁₇N₃O₂S) is a RhoA/Rho kinase (ROCK) inhibitor, it is an isoquinolinesulfonamide derivative having the chemical name hexahydro-1-(5-isoquinolinesulfonyl)-1H-1,4-diazepine[hexahydro-1-(5-isoquinolylcurrent)-1H-1,4-diazepime] or 1-(5-isoquinolinesulfonyl)homopiperazine[1-(5-isovinylethanesulfonyl)homopiperazine], of formula C₁₄H₁₇N₃O₂S. It has been used for the treatment of cerebral vasospasm and has been found to be effective for the treatment of pulmonary hypertension. In the context of the invention, the term “fasudil” means the fasudil molecule and acceptable salts or formulations thereof such as, but not restricted to, CAS numbers 105628-07-7.

In the context of the invention, the term “derivative of fasudil” refers to a compound that is derived from fasudil by a chemical reaction and/or to analogs of fasudil. In a preferred embodiment, the derivative has similar activity to fasudil.

In the context of the invention, the term “neurodevelopmental disorder” means a disorder that affects the development of the nervous system, leading to abnormal brain function which may affect emotion, learning ability, self-control, and memory. In general, the effects of neurodevelopmental disorders tend to last for a person's lifetime. In particular, neurodevelopmental disorders comprise:
Attention deficit/hyperactivity disorder (ADHD),
Communication, speech, or language disorders, including autism spectrum disorder (ASD), expressive language disorder, fluency disorder, social (pragmatic) communication disorder, and speech sound disorder,
Intellectual disabilities (IDs) or intellectual development disorder (IDD, previously called mental retardation) and global developmental delay (GDD),
Motor disorders including developmental coordination disorder, stereotypic movement disorder, and tic disorders (such as Tourette's syndrome),
Neurogenetic disorders, such as fragile-X syndrome, Down syndrome, Rett syndrome, hypogonadotropic hypogonadal syndromes,
Specific learning disorders, like dyslexia or dyscalculia,
Traumatic brain injury (including congenital injuries) and disorders due to neurotoxicants like fetal alcohol spectrum disorder, Minamata disease, behavioral disorders including conduct disorder etc. caused by heavy metals, hydrocarbons, medications, and illegal drugs, like cocaine and others.

In the context of the invention, the term “neuropsychiatric disorder” means disorders of affect, cognition, and behavior that arise from overt disorder in cerebral function, or from indirect effects of extracerebral disease. In particular, neuropsychiatric disorder includes epilepsy, attention deficit/hyperactivity disorder, schizophrenia and bipolar trouble.

### Method of Preventing or Treating a Neurodevelopmental Disorder or a Neuropsychiatric Disorder

The present invention concerns a method of preventing or treating a neurodevelopmental disorder or a neuropsychiatric disorder in a subject, comprising administering a therapeutically effective amount of fasudil or a derivative thereof or a pharmaceutical composition comprising fasudil or a derivative thereof, in a subject in need thereof.

By a “therapeutically effective amount” is meant a sufficient amount of fasudil to treat or prevent a specific disease, at a reasonable benefit/risk ratio applicable to any medical treatment. It will be understood, however, that the total daily usage of fasudil will be decided by the attending physician within the scope of sound medical judgment. The specific therapeutically effective dose level for any particular subject will depend upon a variety of factors including the disorder being treated and the severity of the disorder, activity of fasudil, the age, body weight, general health, sex and diet of the subject, the time of administration, route of administration and rate of excretion of fasudil, the duration of the treatment, drugs used in combination or coincidental with fasudil, and like factors well known in the medical arts.

The terms “pharmaceutical composition” as used herein refer to a compound or composition capable of inducing a desired therapeutic effect when properly administered to a patient.

Such pharmaceutical compositions may comprise a therapeutically effective amount of fasudil, in admixture with a pharmaceutically acceptable carrier selected for suitability with the mode of administration.

“Pharmaceutically” or “pharmaceutically acceptable” refers to molecular entities and compositions that do not produce an adverse, allergic or other untoward reaction when administered to a mammal, especially a human, as appropriate.

In the context of the invention, the term “pharmaceutically acceptable carrier” may be any known pharmaceutical support used for the administration of a molecule to a human or animal, depending on the subject to be treated. In particular, a pharmaceutically acceptable carrier refers to a non-toxic solid, semi-solid or liquid filler, diluent, encapsulating material or formulation auxiliary of any type.

As used herein, “pharmaceutically acceptable carriers” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, dextrose, glycerol, ethanol, and the like, as well as combination thereof. In many cases, it will be preferable to include isotonic agents, such as sugars, polyalcohols, or sodium chloride in the composition and formulation may also contain an antioxidant and a stabilizing agent.

Preferably the pharmaceutical composition comprising fasudil, comprises a therapeutically effective amount of fasudil

In a particular embodiment of the invention, the subject has a disorder selected among intellectual disability, autism spectrum disorder, epilepsy, attention deficit/hyperactivity disorder, schizophrenia and bipolar trouble, more preferably the subject has an autism spectrum disorder.

In a particular embodiment, the subject has hyperactivity, repetitive behaviors and/or deficit in object memory. These symptoms are well defined and known in the art.

Deletions and duplications of the 16p11.2 locus is associated with autism spectrum disorder, intellectual disability, epilepsy, attention deficit hyperactivity disorder, schizophrenia, and bipolar disorder.

In a particular embodiment, the subject suffers from a disorder associated with the rearrangement or the deletion of the 16p11.2 locus and in particular of the 16p11.2 BP4-BP5 region.

Fasudil is a RHOA inhibitor, however in an embodiment of the invention, the subject does not exhibit an increased level of RHOA in the hippocampal region, i.e. the subject has not a level of RHOA in the hippocampal region of the brain significantly superior to the average level in a comparable population. By “comparable population”, it is meant a population of same sex and same age range.

According to an embodiment of the invention, the method is used to improve cognition. By “cognition” it is meant the mental action or process of acquiring knowledge and understanding through thought, experience, and the senses. It encompasses many aspects of intellectual functions and processes such as: attention, the formation of knowledge, memory and working memory, judgment and evaluation, reasoning and “computation”, problem solving and decision making, comprehension and production of language. Cognitive processes use existing knowledge and generate new knowledge.

In the context of the invention, the term “improves cognition” refers to the improvement of these functions and processes, in particular by increasing the speed of these processes or the quantity of knowledge and understanding that are involved in these functions and processes.

In particular, the method according to the invention is used to improve novel object recognition, i.e. analysis, formation of knowledge and memory of objects.

The form of the pharmaceutical compositions, the route of administration, the dosage and the regimen naturally depend upon the condition to be treated or prevented, the severity of the illness, the age, weight, and sex of the patient, etc.

According to the invention, fasudil or the derivative thereof can be administered by any suitable route, in particular by parenteral, or oral (e.g., buccal, inhalation, nasal and pulmonary spray) route.

In a preferred embodiment, fasudil or the derivative thereof is administered regularly and more preferably on a daily basis.

### Use of Fasudil for Preventing or Treating a Neurodevelopmental Disorder or a Neuropsychiatric Disorder

The present invention also concerns the use of fasudil or a derivative thereof for preventing or treating a neurodevelopmental disorder or a neuropsychiatric disorder in a subject.

In a particular embodiment, the subject has a disorder selected among intellectual disability, autism spectrum disorder, epilepsy, attention deficit/hyperactivity disorder, schizophrenia and bipolar trouble, more preferably the subject has an autism spectrum disorder.

In a particular embodiment, the subject suffers from hyperactivity, repetitive behaviors and/or deficit in object memory. These symptoms are well defined and known in the art.

In a particular embodiment, the subject suffers from a disorder associated with the rearrangement or the deletion of the 16p11.2 locus and in particular of the 16p11.2 BP4-BP5 region.

In a particular embodiment of the invention, the subject does not exhibit an increased level of RHOA in the hippocampal region, i.e. the subject has not a level of RHOA in the hippocampal region of the brain significantly superior to the average level in a comparable population. By “comparable population”, it is meant a population of same sex and same age range.

According to an embodiment, the use according of the invention is to improve cognition. The terms “cognition” and “improve cognition” being as defined in the section “Method of preventing or treating a neurodevelopmental disorder or a neuropsychiatric disorder”.

In particular, the use according to the invention is to improve novel object recognition, i.e. analysis, formation of knowledge and memory of objects.

According to the invention, fasudil or the derivative thereof can be administered by any suitable route, in particular by parenteral, or oral (e.g., buccal, inhalation, nasal and pulmonary spray route.

In a preferred embodiment, fasudil or the derivative thereof is administered regularly and more preferably on a daily basis.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

The present invention will be further illustrated by the figures and examples below.

### FIGURES

FIG. 1. Representation of the behavioral pipeline used for investigating the therapeutic effects of fasudil drug in Kctd13^{+/-} and 16p11.2 Del/+ mouse models. 3 cohorts of animals for each genetic line were used. 12-week-old mice were subjected to different behavior and learning tests, after a previous habituation to the phenotyping zone. At 14 weeks, each cohort was divided into two groups. The first group started the fasudil treatment and the second group followed the cognitive characterization without treatment. NOR: novel object recognition.

FIGS. 2(A) and 2B. FIG. 2(A) shows results from the open field test, and FIG. 2(B) shows results from the novel object recognition test of the Kctd13^{+/-} and the 16p11.2 Del/+ mouse models at 12 and 13 weeks of age. As shown in FIG. 2(A) mutant mice from the Kctd13^{+/-} line (wt (n=20) and Kctd13^{+/-} (n=19)) showed no alterations in exploratory (total distance) and vertical activity (rears) compared to their wt littermate whereas the mutant mice from the 16p11.2 Del/+ line (wt (n=38) and Del/+ (n=32)) showed increase exploratory activity in the distance travelled compared to wt. FIG. 2(B) depicts that in the NOR test, the Kctd13^{+/-} and the Del/+ animals (wt (n=21) and Kctd13^{+/-} (n=23) littermates; and wt (n=27) and Del/+ (n=30) littermates) showed a poor object recognition memory compared to their respective wt littermates. (*p<0.05; **p<0.01; ***p<0.001).

FIGS. 3(A) and 3(B). FIG. 3(A) shows results from the open field test, and FIG. 3(B) shows results from the novel object recognition test of the Kctd13^{+/-} and the 16p11.2 Del/+ mouse models with or without fasudil treatment. FIG. 3(A) depicts that kctdl3-deficient mice from the Kctd13^{+/-} line (non treated wt (n=11), treated wt (n=9), non-treated Kctd13^{+/-} (n=10) and treated Kctd13+/− (n=9)) showed no alterations in the different variables compared to their wt littermate. The heterozygous mice from the 16p11.2 Del/+ line (non treated wt (n=20), treated wt (n=18), nontreated Del/+ (n=15) and treated Del/+ (n=14)) showed increased exploratory activity in the distance travelled, which was not affected by the fasudil treatment. The treatment did not have either effect for any genotype on the vertical activity. FIG. 3(B) depicts results from the the NOR test, in which the mutant animals from the Kctd13^{+/-} (non-treated wt (n=12), treated wt (n=11), non-treated Kctd13^{+/-} (n=14) and treated Kctd13^{+/-} (n=10)) or the 16p11.2 Del/+ model (non-treated wt (n=17), treated wt (n=9), non-treated Del/+ (n=13) and treated Del/+ (n=13)) were challenged to recognize the new object from the familiar object. Kctd13^{+/-} and the Del/+ mutant mice were both impaired to recognize the new object compared to their respective wt littermates in the non-treated group and the fasudil treatment was able to restore the object recognition in the Kctd13^{+/-} line and in the 16p11.2 Del/+ model (*p<0.05; **p<0.01; ***p<0.001).

FIGS. 4(A) and 4(B). Detection of RHOA by western blots in heterozygous Kctd13+/− FIG. 4(A) and the 16p11.2 Del/+ FIG. 4(B) hippocampal lysates and their control (wt) littermate. The quantification of the western blot revealed no changes in RHOA protein levels in the Kctd13+/− (FIG. 4(A)) or in the 16p11.2 Del/+ (Fig. (B) mice compared to their wt littermates. Fasudil treatment did not cause changes in RHOA protein levels in the two mutant lines (non-treated wt (n=22), treated wt (n=11), non-treated Kctd13+/− (n=21) and treated Kctd13+/− (n=7); and non-treated wt (n=17), treated wt (n=8), non-treated Del/+ (n=11) and treated Del/+ (n=11)).

FIGS. 5(A) and 5(B). Detection of the phosphorylated form of MLC (P-MLC, A,B) by western blots in heterozygous Kctd13+/− (FIG. 5(A)) and the 16p11.2 Del/+ (FIG. 5(B)) hippocampal lysates and their control (wt) littermate. Kctd13 deficient mice showed an increase in the levels of phosphorylated MLC protein (FIG. 5(A)) and the loss of a copy of 16p11.2 region caused an increase in the levels of phosphorylated MLC protein (FIG. 5(B)). The treatment with fasudil reversed this alteration in Kctd13^{+/-} (non-treated wt (n=21), treated wt (n=10), non-treated Kctd13^{+/-} (n=23) and treated Kctd13^{+/-} (n=9)) and in the Del/+ mutant line (non-treated wt (n=17), treated wt (n=8), non-treated Del/+ (n=14) and treated Del/+ (n=10)). (*p<0.05; **p<0.01).

### EXAMPLE

The inventors made the hypothesis that the deletion of the 16p11.2 region led to overactivation of the RHOA pathway due to the decrease in the number of copies of KCTD13. Overactivation of the RHOA/ROCK pathway would cause behavioral and learning alterations in 16p11.2 deletion syndrome. They tested the fact that the inhibition of this pathway could improve the associated phenotypes in models Kctd13 heterozygous or carrying the deletion of the segment homologous to 16p11.2. The treatment of these models with fasudil (HA1077), an inhibitor of ROCK kinase dependent on the RHOA pathway counterbalanced the memory disorders measured in these models. This demonstrates the relevance of targeting the RHOA pathway as a therapeutic approach for the treatment of cognitive disorders associated with the deletion of the 16p11.2 region and in other diseases inducing ASD.

Materials and Methods

Mouse Lines, Genotyping

Two mice models were used in the study. The 16p11.2 mouse model corresponds to the Del(7Sult1a1-Spn)6Yah mouse model (Arbogast et al., 2016), noted here Del/+. The line was kept on a pure C57BL/6N (B6N) inbred genetic background. The deletion allele was identified by PCR using primers Fwd1 (5′-CCTGTGTGTATTCTCAGCCTCAGGATG-3′) and Rev2 (5′-GGACACACAGGAGAGCTATCCAGGTC-3′) to detect a specific band of 500 bp while the wild-type allele was identified using Fwd1 and Rev1 (5′-GGACACACAGGAGAGCTATCCAGGTC-3′) primers to detect the presence of a 330 bp fragment. PCR program was: 95° C./5 min; 35×(95° C./30 s, 65° C./30 s, 70° C./1 min), 70° C./5 min. The Kctd13^{em2(IMPC)Ics} knock-out mouse was generated by the CRISPR/Cas9 technology (Birling et al., 2017) in the B6N genetic background. Two pairs of sgRNAs, one pair located upstream and the other pair downstream of the target region, were selected to delete the exon 3 and 4 of the gene. Both pairs of sgRNAs (showing a cut) and Cas9 mRNA were microinjected in fertilized eggs derived from super-ovulated sexually immature B6N female mice (4-5 weeks olds). Injected embryos cultured in vitro were implanted into the oviducts of pseudo-pregnant females. The deletion of Kctd13 in Kctd13^{em2(IMPC)Ics} (noted here Kctd13^{+/-}) was confirmed by PCR using primers Ef (5′-ACCTCTTAGCTGGGCATGCTAAATT-3′) and Xr (5′-AGCCTATGCTAACTATTATCACAGG-3′) and the sequence of the deleted fragment. PCR reaction gave deletion and wild-type products of 429 and 668 bp long respectively. PCR program was: 94° C./5 min, 35× (94° C./30 sec; 60° C./30 sec; 72° C./30 sec), 72° C./5 min. This set of primers was also used for genotyping.

Chronic Fasudil Treatment

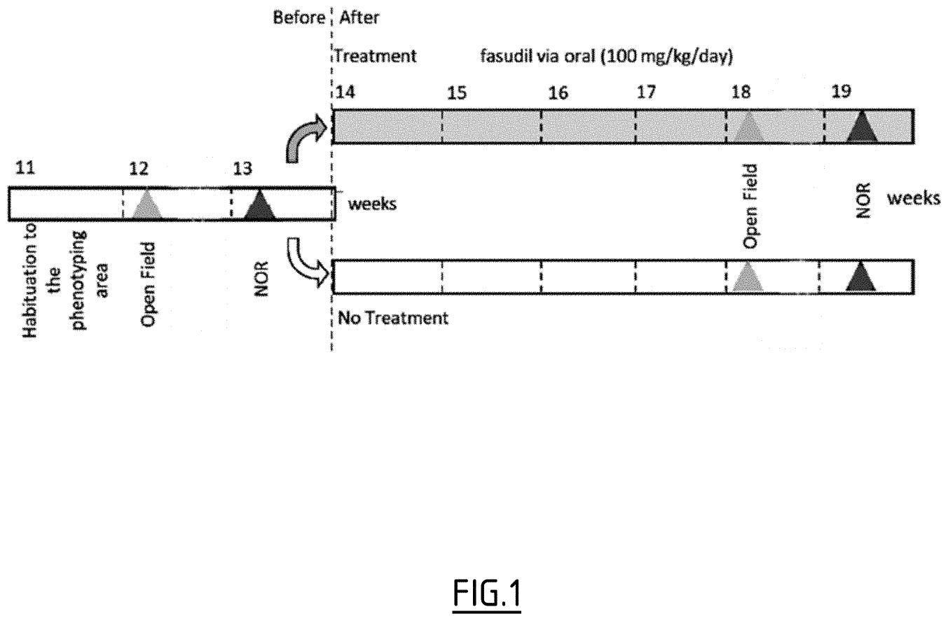

In this study, the inventors developed a protocol for a pre-clinical treatment (FIG. 1) with the drug fasudil hydrochloride or HA1077 (F4660, LC laboratories Boston, Mass., USA). At weaning, control wild type littermates and heterozygous male mice, either Kctd13^{+/-} or Del/+, were taken from several litters and housed in groups of 4-2 individuals in ventilated cages, where they had free access to water and diet. Animal bedding was changed once a week. At 11 weeks, animals were transferred from the animal facility to the phenotyping area. The temperature was kept at 21±2° C., and the light cycle was controlled as 12 h light and 12 h dark.

At 12 weeks of age, three independent cohorts of mice for each line, with wild-type (wt) and mutant littermates, were subjected to a battery of behavioral tests (see below) for 2 weeks. Then, 14 weeks old mice were randomly divided into 2 groups: one treated with fasudil administrated orally ad libitum in drinking water to reach a dose of 100 mg/kg/day and a second with no treatment. The dose was estimated to 152.7 mg in feeding bottle (250 ml) changed twice a week considering the previous study in which the drinking volume was evaluated to about 4.6 ml/day/mouse. Four weeks after the beginning of the treatment, 18-week-old mice were challenged once again to the same battery of behavioral tests (see below) and were kept under the same treatment condition (FIG. 1). All animals injured by their cage companions were excluded from the behavioral tests at the time when they were seen. A second batch of three independent cohorts were processed similarly but without behavior test for the molecular analysis of the hippocampal region from treated and non-treated mice. In this case, treated animals started the fasudil treatment at the age of 12 weeks, for 6 weeks. Samples were quickly harvested from 18 weeks-old mice after euthanasia by cervical dislocation and snap frozen for molecular analyses.

Behavioral analysis

Two tests that previously unraveled robust phenotypes in the three 16p11.2 mice models were used: the open field for the exploration activity, and the novel object recognition for the learning and memory in mice. For the open field (OF) mice were tested in automated arena made of PVC with transparent walls and a black floor and covered with translucent PVC. The arena was divided into central and peripheral regions and homogeneously illuminated at 150 Lux. Each mouse was placed on the periphery of the open field and allowed to explore the apparatus freely for 30 min as a new environment. During each session the exploration activity was analyzed by measuring the total distance travelled, evaluated the adaptation of the mice to the environment over time, by splitting the data in intervals of 10 minutes and assessed the vertical activity through the number of rears. The novel object recognition (NOR) memory task is based on the innate preference of rodents to explore novelty involving the perirhinal and entorhinal cortex and the hippocampus. The test was performed in a circular open field of PVC white with opaque walls and floor of 30 cm high and 50 cm diameter. On the first and second days, each mouse was habituated to the arena for 15 minutes at 60 Lux. The following day, we started the NOR sessions. First, each animal was individually given a 10 minutes acquisition trial for the presentation of two identical objects A (either marble or dice) placed at the northeast or northwest of the open field arena. The exploration time of both objects A was recorded. 3 hours later (retention delay in home cages), a 10 minutes retention trial (second trial) was performed. One of the identical object A was replaced with a novel object B at the same position. The exploration time of the two objects (familiar object and novel object) was recorded. The recognition index (RI) was defined as (tB/(tA+tB)×100). A RI of 50% corresponds to chance level and a significantly higher RI reflects good recognition memory. All mice that did not explore the objects for more than 3 seconds during the acquisition trial or the retention trial or did not show any interest for one object were excluded from the analysis.

Western Blot

Fresh hippocampal tissues were isolated by rapid decapitation/dissection of naive mice and snap frozen. Then, they were lysed in ice-cold sonication buffer supplemented with Complete™ Protease Inhibitor Cocktail (Roche). Individual samples were disaggregated, centrifuged at 4° C. for 30 minutes at 14000 rpm, diluted in 4× Laemmli sample buffer containing β-mercaptoethanol (Bio-Rad, France), and incubated at 95° C. for 5 min. Protein concentration was determined by Pierce™ BCA Protein Assay Kit (23225, Thermo Fisher Scientific, Strasbourg). Samples were diluted with sample buffer such that 30 μg of protein were loaded per lane onto 15% polyacrylamide gel. Gels were run and then transferred to nitrocellulose membranes by Trans-Blot® Turbo™ Transfer System (BioRad, France) through MIXED MW Bio-Rad Preprogrammed Protocol. Then they were blocked in 5% BSA, 1× (TBS-T) and incubated with primary antibody during 10 minutes. Membranes were washed in TBS-T followed by a 10 minutes secondary antibody incubation using an HRP conjugated Goat anti-Rabbit IgG (A16096, Invitrogen, France) at 1:5,000 through SNAP i.d.® 2.0 Protein Detection System (C73105, Merck). This apparatus has a vacuum-driven technology and a built-in flow distributor that actively drives reagents through the membrane. Total levels of RHOA protein and Myosin Light Chain phosphorylation by Myosin Light Chain Kinase via RHOA pathway were analyzed using Western Blot. Proteins were visualized with Amersham™ Imager 600. Signals were quantified using ImageJ and analyzed using Microsoft Excel and GraphPad Prism. We used the following primary antibodies: RHOA (2117, Cell Signaling, USA, 1:1,000) and pMLC (Thr18/Ser19 #3674, Cell signaling, Boston, Mass., USA, 1:1,000). The ratio of protein level or phosphorylation level against control β-actin protein level (detected with a mouse monoclonal Anti-βActin-Peroxidase antibody (A3854 Sigma)) was normalized to untreated wt sample mean.

Statistical Analysis

The statistical analysis was carried out using standard statistical procedures available on the SigmaPlot software (Systat software, San Jose, USA). All outliers were identified using the Grubbs' test from calculator GraphPad (GraphPad Software, San Diego) or ROUT method with a Q value of 1% from GraphPad Prism 7.01 (GraphPad Software, San Diego) when data with nonlinear regression. Data from behavioral characterization of Kctd13^{+/-} and 16p11.2 Del/+ mouse models were analyzed through the Student t-test (see supplementary table 1 and table 2 as a summary). One sample t-test was used to compare recognition index values to the set chance level (50%). Data from posttreatment behavioral phenotyping of both genetic lines were analyzed using one-way ANOVA followed by Tukey's post-hoc test whenever data presented normal distribution and equal variance. Otherwise, the non-parametric Kruskal-Wallis one-way analysis of variance and Mann-Whitney U test were used. One sample t-test was used also to compare recognition index values to the set chance level (50%). Western blot data were analyzed using Kruskal-Wallis one-way analysis of variance test between groups followed by Mann-Whitney Utest or Student t-test depending on data distribution. Data are represented as the mean±SEM and the significant threshold was p<0.05.

Results

Phenotypic Characterization of the New Kctd13+/− Mouse Model and Comparison with the 16p11.2 Del/+ Mouse Model

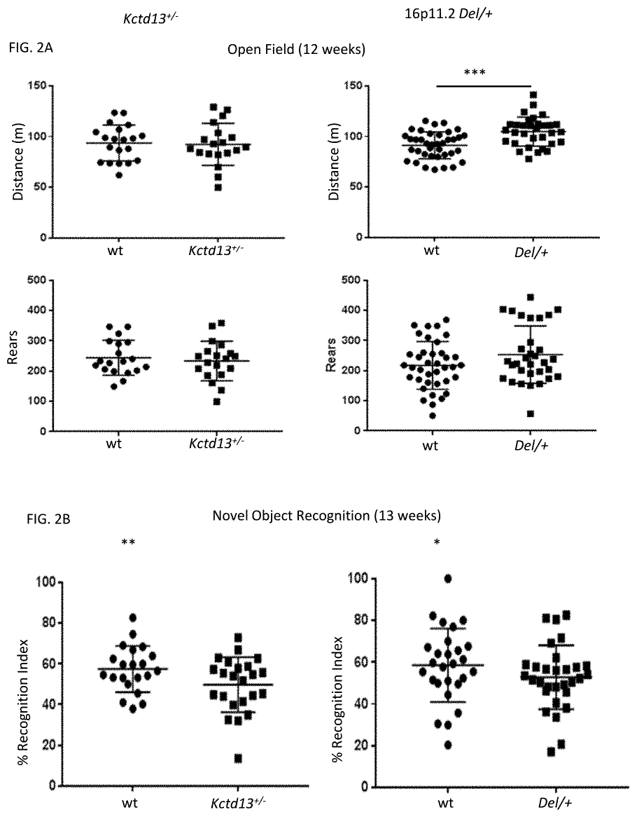

A new Kctd13 KO mouse model was created with the deletion of exon 3 and 4, and we evaluated its behavior in two tasks and compared the results obtained with the characterization of 16p11.2 Del/+ model (FIGS. 2A and 2B). First, the inventors did not detect significant differences in the open field test (12 weeks), when measuring the total distance travelled by Kctd13+/− mice compared to wt littermates. This result was different from the phenotype of the Del/+ mice (FIG. 2A). Indeed Del/+ mutant mice were more active with a significant increase in the distance travelled compared to their wt littermates (Student t test: Total distance: wt vs. Del/+ t(68)=−4.096; p<0.001). Then, the distance travelled in 5-minute intervals was analyzed, to see the habituation to a new environment during the test. The Kctd13+/− mice experienced a similar habituation to the control individuals. Here too, a significant difference was seen in the 16p11.2 Del/+ carrier mice. Although being more active, the mutant mice showed normal habituation compared to their wildtype littermates. Finally, the vertical activity with the numbers of rears was evaluated, and the inventors did not see any significant differences in the two lines. However, the Del/+ mice developed a tendency to the appearance of repetitive behavior, measured from the number of rears with 8 Del/+ animals having a strong rearing activity but as a group it was not significant.

Finally, the inventors investigated whether Kctd13^{+/-} mice could discriminate a novel object from a previously explored set of two objects after a retention delay of 3 hours in the NOR task (FIG. 2B). Whereas wt animals were able to differentiate objects showing a novel object preference, Kctd13^{+/-} mice were not able to discriminate the novel from the familiar object. The deficit was like the one seen in the 16p11.2 Del/+ mice (One sample t-test for the Kctd13^{+/-} model: wt (t(20)=3.0179; p=0.0068), Kctd13^{+/-} (t(22)=0.0805; p=0.9366); and the 16p11.2 Del/+ model: wt (t(26)=2.5312; p=0.0178), Del/+ (t(29)=1.0119; p=0.3199)). Overall, behavioral analysis showed that the Kctd13 haploinsufficiency in the pure C57BL/6N genetic background phenocopied recognition memory deficits seen in the 16p11.2 deletion model. However, the increased exploration activity found in the 16p11.2 Del/+ mice was not seen in the Kctd13+/− mutant mice.

Fasudil treatment partially reverses the cognitive impairment in the Kctd13^{+/-} and in the 16p11.2 Del/+ mice models

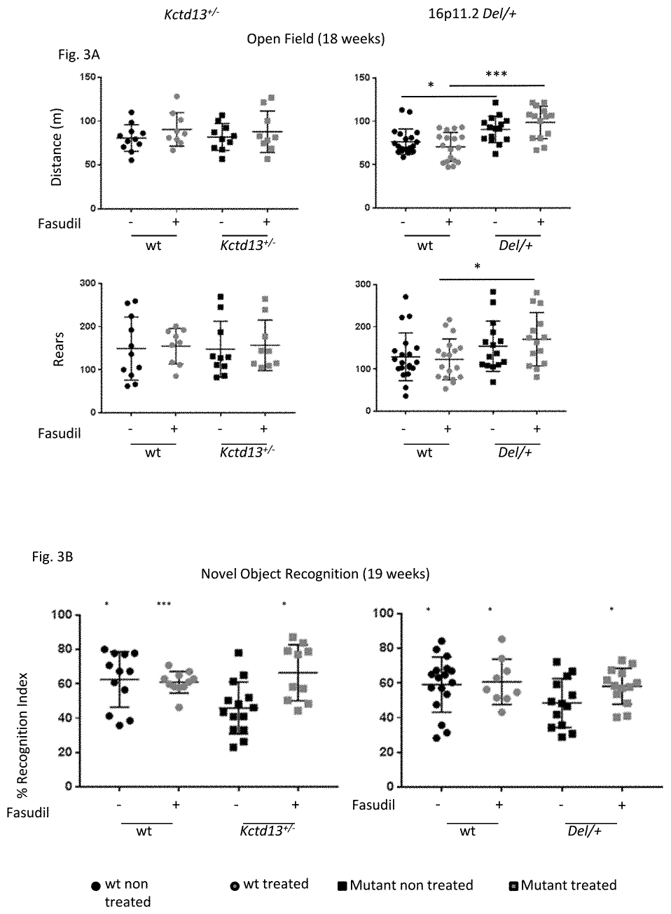

Thus, after the behavioral characterization of the Kctd13^{+/-} and 16p11.2 Del/+ mouse models, both genotypes (wt and mutant) were subdivided into two groups. Individuals were randomly assigned to a group that was treated with fasudil or an untreated control non-treated group prior to further behavior testing (FIG. 1). First, the fasudil treatment did not change the locomotor exploration activity of the different genotypes (FIG. 3A) with Del/+ individuals travelling more than the control wt with no effect of the treatment (One way ANOVA between groups: F(3,63)=10.158; p<0.001; Tukey's post hoc tests: non treated wt vs. treated wt: p=0,675, non-treated Del/+ vs. treated Del/+: p=0.54, non-treated wt vs. treated Del/+: p=0.001, treated wt vs. non treated Del/+: p=0.004 and treated wt vs. treated Del/+: p<0.001). The treatment did not have effect on any genotype for the vertical activity (Kruskal-Wallis one-way analysis of variance: H(3)=7.074; p=0.070). However, fasudil treatment significantly improved the NOR defect found in both Kctd13^{+/-} (One sample t-test: non-treated wt (t₍₁₁₎=2.6929; p=0.0209), treated wt (t₍₁₀₎=5.7297; p=0.0002), non-treated Kctd13^{+/-} (t₍₁₁₎=2.6101; p=0.0243 (less than 50%), treated Kctd13^{+/-} (t₍₉₎=3.1937; p=0.0109)) and 16p11.2 Del/+ mice ((One sample t-test: nontreated wt (t₍₁₆₎=2.3736; p=0.0305), treated wt (t₍₈₎=2.4481; p=0.0401), non-treated Del/+ (t₍₁₂₎=0.3787; p=0.7115), treated Del/+ (t₍₁₀₎=2.9168; p=0.0154)) one week later. This observation confirmed the altered recognition memory in mutants at 19 weeks, detected previously at 12 weeks, and showed the protective effect of fasudil treatment in the Kctd13^{+/-} and the 16p11.2 Del/+ models (FIG. 3B).

Molecular analyses of RHOA/ROCK signaling pathway in the Kctd13^{+/-} and the 16p11.2 Del/+ mouse models

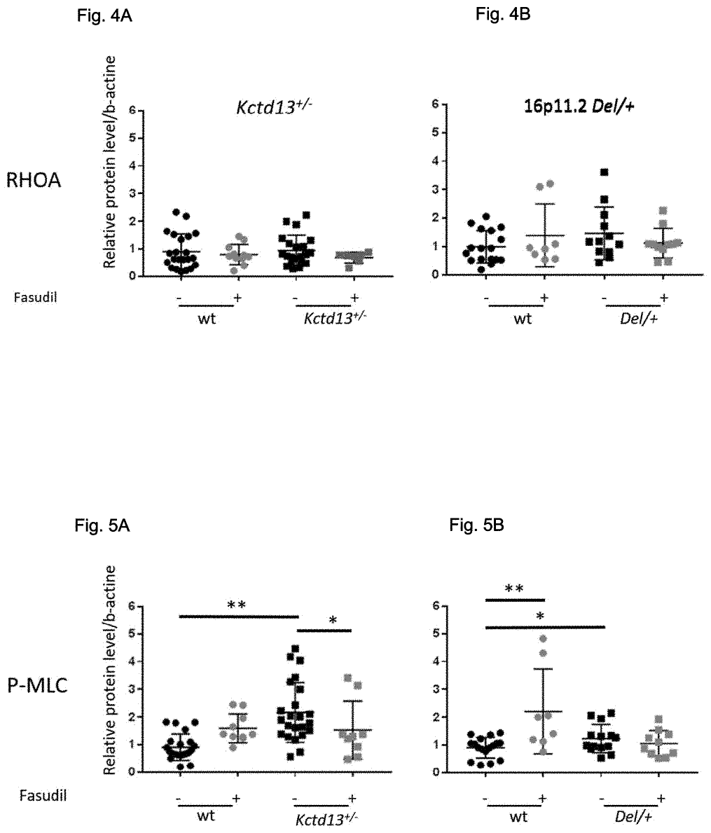

Then the inventors checked whether the RHOA/ROCK signaling pathway was changed in both 16p11.2 deletion and Kctd13 mouse models. Neither the loss of one copy of the complete chromosomic region in 16p11.2 Del/+ mice nor the deficiency of Kctd13 resulted in increased levels of RHOA in the hippocampal region at 18 weeks of age (FIG. 4A and B). Nevertheless, both models showed an over activation of the RHOA/ROCK pathway. Indeed, Myosin Light Chain (MLC), a protein targeted by the RHOA/ROCK pathway, showed increased levels of phosphorylation in the hippocampus of these mice, supporting the idea that this pathway could be associated to the cognitive phenotype observed (FIG. 5A and B). Therefore, the inventors verified whether fasudil therapeutic effect was acting through the inhibition of this RHOA/ROCK signaling pathway. Interestingly, the fasudil treatment reduced MLC phosphorylation levels in Kctd13 mutant individuals (Kruskal-Wallis one-way analysis of variance between groups: H(₃)=21.731; p<0.001; Mann-Whitney Test: non treated wt vs. non treated Kctd13^{+/-}: p=0.009; non treated wt vs. treated Kctd13^{+/-}: p=0.702; non treated Kctd13+/− vs. treated Kctd13^{+/-}: p=0.049). As for the Kctd13^{+/-} model, in 16p11.2 deficient mice fasudil restored a normal MLC phosphorylation in treated mutant mice but surprisingly induced increased MLC phosphorylation in wt mice (Kruskal-Wallis one way analysis of variance between groups: H(3)=8.457; p=0.037; Mann-Whitney Test: non treated wt vs. treated wt: p=0.008; t-test: non treated wt vs. non treated Del/+: p=0.047, non-treated wt vs. treated Del/+: p=0.364).

Discussion

Here, the phenotypes of another Kctd13^{+/-} mouse mutant line that replicates some of the defects seen in young mouse models carrying the 16p11.2 BP4-BP5 deletion is described and a treatment aiming at reducing the activity of the RHOA/ROCK pathway on cognition was explored. With the new Kctd13^{+/-} mouse line, the inventors were able to detect changes in the NOR but no effect on the exploration activity compared to the 16p11.2 Del/+ mouse model. These results settle with a recent study (Arbogast et al., 2019) highlighting the role of KCTD13 in the 16p11.2 deletion syndromes. The loss of one copy of Kctd13 gene did not cause alterations on exploration or vertical activity of mice in open field test. However, the hemi-deletion of entire 16p11.2 region induced hyperactivity in these animals. This observation leads us to propose that Kctd13 genetic dosage is not involved in the increased exploration activity associated with 16p11.2 deletion. Accordingly, chronic fasudil administration did not attenuate the hyper locomotion affecting 16p11.2 Del/+ mice. This finding suggests that there could be other genes of the region involved in this phenotype. For this reason, the treatment with an inhibitor of the RHOA/ROCK pathway, deregulated because of Kctd13 decreased levels in 16p11.2 deficient, did not produce any effect.

The object recognition memory deficit is one of the most robust and reproducible phenotypes associated with 16p11.2 Del/+ mice (Arbogast et al., 2016). In agreement with precedent research, the Kctd13+/− mouse model has deficits in novelty detection in NOR. Furthermore, fasudil treatment significantly rescued this impairment in mutant mice. Thus, loss of KCTD13 is a driver of object recognition phenotype associated with 16p11.2 deletion. In addition, the therapeutic effects of fasudil on Kctd13 deficient and 16p11.2 deletion mice highlight the role of the RHOA/ROCK signaling pathway as the main mechanism responsible of this phenotype.

Interestingly deficient individuals for the Kctd13 gene showed no change in expression levels of the RHOA protein and the fasudil treatment did not modify RHOA protein expression in mutant and control mice. Likewise, the carriers of the 16p11.2 hemi-deletion did not display changes of expression for this protein. A change in the RHOA protein level was not detected, however Kctd13 mutants as well as 16p11.2 deficient mice had increased phosphorylated-MLC levels. This observation confirms that RHOA/ROCK pathway outcome is over-activated due to a loss copy of Kctd13 in Kctd13 and 16p11.2 mutants. Furthermore, the therapeutic effect of fasudil in recognition memory phenotype associated to 16p11.2 CNV was due to the normalizing action of the drug in both mouse models. These results highlight the clinical relevance of treatment because of its potential as a cognitive enhancer in humans with memory and learning dysfunction related to neurodevelopmental disorders.

### CONCLUSION

Kctd13 haploinsufficiency phenocopied the recognition memory deficits seen in the 16p11.2 deletion model but did not change the exploration activity in the open field. Treatment in adult mice with fasudil, an inhibitor of RHOA, was able to restore novel object recognition memories. Furthermore, fasudil treatment was able to restore RHOA/ROCK signaling pathway to almost normal level of phosphorylation of the Myosin Light Chain in the brain. Altogether targeting the RHOA pathways may be an alternative for improving cognition in people carrying the 16p11.2 deletion.

### REFERENCES

Arbogast, T., et al., 2016. Reciprocal Effects on Neurocognitive and Metabolic Phenotypes in Mouse Models of 16p11.2 Deletion and Duplication Syndromes. PLoS Genet. 12, e1005709

Birling, M. C., et al., 2017. Efficient and rapid generation of large genomic variants in rats and mice using CRISMERE. Sci Rep. 7, 43331.

1. A method of preventing or treating a neurodevelopmental disorder or a neuropsychiatric disorder in a subject, comprising administering a therapeutically effective amount of fasudil, or a derivative thereof, or a pharmaceutical composition comprising fasudil or a derivative thereof, to a subject in need thereof.

2. The method according to claim 1, wherein said disorder is selected among intellectual disability, autism spectrum disorder, epilepsy, attention deficit/hyperactivity disorder, schizophrenia and bipolar trouble.

3. The method according to claim 1 wherein the subject suffers from hyperactivity, repetitive behaviors and/or deficit in object memory.

4. The method according to claim 1 wherein the subject has an autism spectrum disorder.

5. The method according to claim 1 wherein the subject suffers from a disorder associated with the rearrangement or the deletion of the 16p11.2 locus.

6. The method according to claim 1 wherein the subject does not exhibit an increased level of RHOA in the hippocampal region.

7. The method according to claim 1, wherein the administration improves cognition.

8. The method according to claim 7, wherein the administration improves novel object recognition.

9. The method according to claim 1, wherein the fasudil or the derivative thereof is administered orally.

10. The method according to claim 1, wherein the fasudil or the derivative thereof is administered on a daily basis.

11. The method according to claim 1, wherein the subject is a human.

12. The method according to claim 1, wherein the fasudil is administered in the form of a pharmaceutically acceptable salt.

13. A pharmaceutical composition for preventing or treating a neurodevelopmental disorder or a neuropsychiatric disorder in a subject, comprising fasudil or a derivative thereof.

14. A pharmaceutical composition according to claim 13 comprising a pharmaceutically acceptable carrier.
